(19) United States
(12) Patent Application Publication
(10) Pub. No.: US 2025/0017840 A1
(43) Pub. Date: Jan. 16, 2025

(54) NOVEL SENOTHERAPY PEPTIDE AND COMPOSITION CONTAINING THE SAME

(71) Applicant: AMOREPACIFIC CORPORATION, Seoul (KR)

(72) Inventors: Hyuk KIM, Yongin-si (KR); Sunyoung PARK, Yongin-si (KR); Si Young CHO, Yongin-si (KR); Sang Hyun MOH, Seongnam-si (KR); Jeong Hun LEE, Seoul (KR); Hyo Hyun SEO, Incheon (KR); Hye In KIM, Incheon (KR)

(73) Assignee: AMOREPACIFIC CORPORATION, Seoul (KR)

(21) Appl. No.: 18/731,949

(22) Filed: Jun. 3, 2024

(30) Foreign Application Priority Data
Jul. 14, 2023 (KR) 10-2023-0091814

(51) Int. Cl. A61K 8/64; A23L 33/18; A61Q 19/08; C07K 14/00

(52) U.S. Cl. CPC A61K 8/64; A23L 33/18; A61Q 19/08; C07K 14/001

(57) ABSTRACT

A novel senotherapy peptide and a method for senotherapy are disclosed. The method for sentherapy includes administering an effective amount thereof to a subject in need thereof. The novel senotherapy peptide suppresses the interaction between p53 and FOXO4 in the nucleus due to a better binding force with p53 than FOXO4, thereby allowing p53 to be released out of the nucleus and move to mitochondria to induce apoptosis of senescent cells and selectively induce the apoptosis of senescent cells in the skin cells.

### CROSS REFERENCE TO RELATED APPLICATION

The present application claims the priority of Korean Patent Application No. 10-2023-0091814 filed on Jul. 14, 2023, the entire contents of which is incorporated herein for all purposes by this reference.

### INCORPORATION BY REFERENCE OF SEQUENCE LISTING

The content of the electronically submitted sequence listing, file name: Q298939_Sequence_Listing_ST26; size 4.5 KB; and date of creation: May 28, 2024, filed herewith, is incorporated herein by reference in its entirety.

### BACKGROUND OF THE INVENTION

### Field of the Invention

The present disclosure relates to a novel senotherapy peptide and a composition containing the same.

### Description of the Related Art

Cellular senescence is known to not only contribute to senescence of a tissue but also plays an important role in the incidence of diseases related to deterioration of tissue function. As the senescence progresses, the number of senescent cells with reduced function increases, and the increased senescent cells secrete inflammatory factors called senescence-associated secretory phenotype (SASP) to cause inflammation and disease in the tissue.

Recently, attention has been focused on the development of more effective and safer senotherapeutics, as it has been reported that the senotherapeutics such as senolytic preparations that can selectively remove the senescent cells and senomorphics that can suppress the secretion of SASP to restore function of the senescent cells to normal are treated to suppress the tissue senescence so that it is possible to prolong lifespan or treat geriatric diseases.

The development of these substances has been recognized as the most important target for the development of substances for senotherapy because they can be widely applied to suppress the senescence, prevent deterioration of the senescence-related function, and treat the geriatric diseases. Recently, as it has been reported that various compounds can be used to improve the senescent tissue and treat some diseases, attention has been more increased on senotherapeutics including senolytics.

However, the substances reported to date are drugs developed through screening using the conventional compound libraries, and have been already known as anticancer drugs or have been used as the conventional other drugs, so that the substances are known to have a limitation that side effects may occur when administered to a human body for a long period of time or have a high antioxidant effect. In view of the above, the development of substances that directly identify senotherapy effects and mechanisms is still insufficient.

### SUMMARY OF THE INVENTION

A purpose of the present disclosure is to provide a novel senotherapy peptide that induces apoptosis of skin senescent cells by suppressing the interaction between p53 and FOXO4 (Forkhead box protein 4).

In order to achieve the above purpose, an embodiment of the present invention provides a peptide containing the amino acid sequence of SEQ ID NO: 1.

Other embodiment of the present invention provides a composition for improving skin wrinkles, comprising a peptide containing the amino acid sequence of SEQ ID NO: 1 as an active ingredient.

Another embodiment of the present invention provides a composition for senotherapy, comprising a peptide containing the amino acid sequence of SEQ ID NO: 1 as an active ingredient.

The peptide according to the present disclosure suppresses the interaction between p53 and FOXO4 in the nucleus due to a better binding force with p53 than FOXO4, thereby allowing p53 to be released out of the nucleus and move to mitochondria to induce apoptosis of senescent cells and selectively induce the apoptosis of senescent cells in the skin cells.

Further, the composition comprising the peptide according to the present disclosure as an active ingredient can effectively improve skin wrinkles through excellent MMP-1 suppressive activity.

Furthermore, the composition comprising the peptide according to the present disclosure as an active ingredient can have an excellent apoptotic effect on selectively senescent cells in the skin cells.

### BRIEF DESCRIPTION OF THE DRAWINGS

FIGS. 1A and 1B show the results confirming that FOXO4 expression increases in skin senescent cells.

FIG. 1C shows relative expression levels of the target gene, with a correction for a GAPDH gene.

FIG. 2 shows the results of confirming an apoptotic efficacy of a peptide according to an embodiment of the present invention in skin senescent cells.

FIGS. 3A and 3B show the results of confirming an apoptotic efficacy of a peptide according to an embodiment of the present invention in skin senescent cells through B-gal stain and microscopic observation.

FIG. 4 shows the results of observing the cytoplasmic movement of p53 by a peptide according to an embodiment of the present invention.

FIG. 5 shows relative expression levels of the target gene, with a correction for a GAPDH gene.

### DETAILED DESCRIPTION OF THE INVENTION

Hereinafter, the present invention will be described in detail.

In an aspect, the present invention may relate to a peptide containing the amino acid sequence of SEQ ID NO: 1.

In other aspect, the present invention may relate to a composition for improving skin wrinkles, comprising a peptide containing the amino acid sequence of SEQ ID NO: 1 as an active ingredient.

In another aspect, the present invention may relate to a method for improving skin wrinkles, comprising administering an effective amount of a peptide containing the amino acid sequence of SEQ ID NO: 1 to a subject in need thereof.

In another aspect, the present invention may relate to a use of a peptide containing the amino acid sequence of SEQ ID NO: 1 for preparing a composition for improving skin wrinkles.

In another aspect, the present invention may relate to a peptide containing the amino acid sequence of SEQ ID NO: 1 for use in improving skin wrinkles.

In another aspect, the present invention may relate to a composition for inducing apoptosis of skin senescent cells, comprising a peptide containing the amino acid sequence of SEQ ID NO: 1 as an active ingredient.

In another aspect, the present invention may relate to a method for inducing apoptosis of skin senescent cells, comprising administering an effective amount of a peptide containing the amino acid sequence of SEQ ID NO: 1 to a subject in need thereof.

In another aspect, the present invention may relate to a use of a peptide containing the amino acid sequence of SEQ ID NO: 1 for preparing a composition for inducing apoptosis of skin senescent cells.

In another aspect, the present invention may relate to a peptide containing the amino acid sequence of SEQ ID NO: 1 for use in inducing apoptosis of skin senescent cells.

In another aspect, the present invention may relate to a composition for senotherapy comprising a peptide containing the amino acid sequence of SEQ ID NO: 1 as an active ingredient.

In another aspect, the present invention may relate to a method for senotherapy, comprising administering an effective amount of a peptide containing the amino acid sequence of SEQ ID NO: 1 to a subject in need thereof.

In another aspect, the present invention may relate to a use of a peptide containing the amino acid sequence of SEQ ID NO: 1 for preparing a composition for senotherapy.

In another aspect, the present invention may relate to a peptide containing the amino acid sequence of SEQ ID NO: 1 for use in senotherapy.

In an embodiment, the senotherapy may include inducing apoptosis of the skin senescent cells.

In an embodiment, the peptide may contain the amino acid sequence of AYSQNGWANRRRRGGRRRRRR (SEQ ID NO: 1).

In an embodiment, the peptide may consist of the amino acid sequence of AYSQNGWANRRRRGGRRRRRR (SEQ ID NO: 1).

In an embodiment, the peptide may suppress the activity of MMP-1.

In an embodiment, the peptide may be contained in an amount of 0.001 to 1% by weight in the composition for improving skin wrinkles.

Specifically, the peptide may be contained in an amount of 0.001% by weight or more, 0.002% by weight or more, 0.003% by weight or more, 0.004% by weight or more, 0.005% by weight or more, 0.006% by weight or more, 0.007% by weight or more, 0.008% by weight or more, 0.009% by weight or more, 0.01% by weight or more, 0.02% by weight or more, 0.03% by weight or more, 0.04% by weight or more, 0.05% by weight or more, 0.06% by weight or more, 0.07% by weight or more, 0.08% by weight or more, 0.09% by weight or more, 0.1% by weight or more, 0.2% by weight or more, 0.3% by weight or more, 0.4% by weight or more, 0.5% by weight or more, 0.6% by weight or more, 0.7% by weight or more, 0.8% by weight or more, or 0.9% by weight or more in the composition for improving skin wrinkles. In addition, the peptide may be contained in an amount of 1% by weight or less, 0.9% by weight or less, 0.8% by weight or less, 0.7% by weight or less, 0.6% by weight or less, 0.5% by weight or less, 0.4% by weight or less, 0.3% by weight or less, 0.2% by weight or less, 0.1% by weight or less, 0.09% by weight or less, 0.08% by weight or less, 0.07% by weight or less, 0.06% by weight or less, 0.05% by weight or less, 0.04% by weight or less, 0.03% by weight or less, 0.02% by weight or less, 0.01% by weight or less, 0.009% by weight or less, 0.008% by weight or less, 0.007% by weight or less, 0.006% by weight or less, 0.005% by weight or less, 0.004% by weight or less, 0.003% by weight or less, or 0.002% by weight or less in the composition for improving skin wrinkles.

In an embodiment, the peptide may be contained in an amount of 0.001 to 1% by weight in the composition for senotherapy or the composition for inducing apoptosis of skin senescent cells.

Specifically, the peptide may contained in an amount of 0.001% by weight or more, 0.002% by weight or more, 0.003% by weight or more, 0.004% by weight or more, 0.005% by weight or more, 0.006% by weight or more, 0.007% by weight or more, 0.008% by weight or more, 0.009% by weight or more, 0.01% by weight or more, 0.02% by weight or more, 0.03% by weight or more, 0.04% by weight or more, 0.05% by weight or more, 0.06% by weight or more, 0.07% by weight or more, 0.08% by weight or more, 0.09% by weight or more, 0.1% by weight or more, 0.2% by weight or more, 0.3% by weight or more, 0.4% by weight or more, 0.5% by weight or more, 0.6% by weight or more, 0.7% by weight or more, 0.8% by weight or more, or 0.9% by weight or more in the composition for senotherapy or the composition for inducing apoptosis of skin senescent cells. In addition, the peptide may be contained in an amount of 1% by weight or less, 0.9% by weight or less, 0.8% by weight or less, 0.7% by weight or less, 0.6% by weight or less, 0.5% by weight or less, 0.4% by weight or less, 0.3% by weight or less, 0.2% by weight or less, 0.1% by weight or less, 0.09% by weight or less, 0.08% by weight or less, 0.07% by weight or less, 0.06% by weight or less, 0.05% by weight or less, 0.04% by weight or less, 0.03% by weight or less, or 0.002% by weight or less in the composition for senotherapy or the composition for inducing apoptosis of skin senescent cells.

In an embodiment, the composition may be a cosmetic or health food composition. Specifically, the composition may be a cosmetic composition for improving skin wrinkles, a health food composition for improving skin wrinkles, a cosmetic composition for inducing apoptosis of skin senescent cells, and a health food composition for inducing apoptosis of skin senescent cells.

A formulation of the food composition is not particularly limited, and may be formulated into, for example, tablets, granules, pills, powders, liquids such as drinks, caramels, gels, bars, tea bags, etc. In addition to the active ingredients, the food composition of each formulation may be appropriately selected from and mixed with ingredients commonly used in the relevant field without difficulty by a person skilled in the art depending on the formulation or the purpose of use, and may have a synergistic effect when applied simultaneously with other raw materials.

The composition may be administered in various ways such as simple ingestion, drinking, injection, spray administration, or squeeze administration.

The food composition according to an embodiment of the present invention may include, for example, various foods such as chewing gum, caramel products, candies, ice cream, and confectionery, beverage products such as soft drinks, mineral water, and alcoholic beverages, health functional food products such as vitamins and minerals, etc.

The food composition according to an embodiment of the present invention may include food additives in addition to the active ingredients. The food additives can generally be understood as substances that are added to, mixed with, or infiltrated into the food when manufacturing, processing, or preserving the food. Since the food additives are consumed with the food daily and for a long period of time, their safety must be guaranteed. In the food additive code of each country that regulates the manufacturing and distribution of food (in Korea, it is the Food Sanitation Act), the food additives that guarantee safety are limited in terms of ingredients or functions. In the Korean Food Additive Code (Notification of Ministry of Food and Drug Safety “Food Additive Standards and Specifications”), the food additives are classified into chemical synthetic products, natural additives, and mixed preparations in terms of the ingredients. These food additives are classified into sweeteners, flavoring agents, preservatives, emulsifiers, acidulants, thickeners, etc., in terms of the functions.

The sweeteners are used to impart an appropriate sweetness to the food, and both natural and synthetic sweeteners may be used in the food composition according to an aspect of the present invention. Preferably, the natural sweetener is used and may include sugar sweeteners such as corn syrup solids, honey, sucrose, fructose, lactose, and maltose.

The flavoring agents are used to improve taste or aroma, and both natural and synthetic flavoring agents may be used. Preferably, the natural flavoring agents are used. When using the natural flavoring agents, they may also serve the purpose of enhancing nutrition in addition to flavoring. The natural flavoring agents may be obtained from apples, lemons, tangerines, grapes, strawberries, peaches, etc., or may be obtained from green tea leaves, Solomon's seals, bamboo leaves, cinnamon, chrysanthemum leaves, jasmine, etc. Also, the natural flavoring agents obtained from ginseng (red ginseng), bamboo shoots, aloe vera, and ginkgo nuts may be used. The natural flavoring agents may be liquid concentrates or solid extracts. In some cases, the synthetic flavoring agents may be used and include esters, alcohols, aldehydes, terpenes, etc.

The preservatives may include calcium sorbate, sodium sorbate, potassium sorbate, calcium benzoate, sodium benzoate, potassium benzoate, EDTA (ethylenediaminetetraacetic acid), etc. The emulsifiers may include acacia gum, carboxymethyl cellulose, xanthan gum, pectin. etc. The acidulants may include citric acid, malic acid, fumaric acid, adipic acid, phosphoric acid, gluconic acid, tartaric acid, ascorbic acid, acetic acid, etc. In addition to improving taste, the acidulants may be added to ensure that the food composition has an appropriate acidity for the purpose of suppressing the growth of microorganisms. The thickeners may include a suspension implementing agent, a precipitating agent, a gel forming agent, a swelling agent, etc.

In addition to the food additives described above, the food composition according to an embodiment of the present invention may comprise physiologically active substances or minerals known in the art and guaranteed to be safe as the food additives for the purpose of supplementing and reinforcing functionality and nutrition.

Such physiologically active substances include catechins contained in green tea, vitamins such as vitamin Bi, vitamin C, vitamin E, and vitamin B12, tocopherol, dibenzoylthiamine, etc., and the minerals include calcium preparations such as calcium citrate, magnesium preparations such as magnesium stearate, iron preparations such as iron citrate, chromium chloride, potassium iodine, selenium, germanium, vanadium, zinc, etc.

The food composition according to an embodiment of the present invention may comprise the above-mentioned food additives in an appropriate amount to achieve the purpose of addition depending on the product type.

Regarding other food additives that may be contained in the food composition according to an embodiment of the present invention, reference may be made to each country's food code or food additive code.

The cosmetic composition according to an embodiment of the present invention may comprise a cosmetically or dermatologically acceptable mediums or bases. They are all formulations suitable for topical application, and may be provided in the form of solution, gels, solids, pasty anhydrous products, emulsion obtained by dispersing an oil phase into a water phase, suspension, microemulsions, microcapsules, microgranules, or ionic (liposomes) and non-ionic vesicular dispersant, or in the form of creams, skins, lotions, powders, ointments, spray, or concealer sticks. This composition can be prepared according to a conventional method in the art. The cosmetic composition may also be used in the form of an aerosol composition further comprising a propellant compressed in the form of a foam.

The cosmetic composition is not particularly limited in its formulation, and may be appropriately selected depending on the purpose. For example, the cosmetic composition may be prepared into the formulations such as skin lotion, skin softener, skin toner, lotion, milk lotion, moisture lotion, nutritional lotion, massage cream, nutritional cream, moisture cream, hand cream, foundation, essence, nutritional essence, pack, soap, cleansing foam, cleansing lotion, cleansing cream, cleansing water, powder, body lotion, body cream, body oil, body cleanser, and body essence.

In case the formulation of the present invention is the paste, the cream or the gel, it may include, as a carrier ingredient, animal fiber, plant fiber, wax, paraffin, starch, tracant, cellulose derivative, polyethylene glycol, silicone, bentonite, silica, talc, zinc oxide, etc.

In case the formulation of the present invention is the powder or the spray, it may include, as a carrier ingredient, lactose, talc, silica, aluminum hydroxide, calcium silicate, or polyamide powder. In particular, in case the formulation is the spray, it may further comprise a propellant such as chlorofluorohydrocarbon, propane/butane or dimethyl ether.

In case the formulation of the present invention is the solution or the emulsion, it may include, as a carrier ingredient, a solvent, a solvating agent, or an emulsifying agent, such as water, ethanol, isopropanol, ethyl carbonate, ethyl acetate, benzyl alcohol, benzyl benzoate, propylene glycol, 1,3-butyl glycol oil, aliphatic ester of glycerol, polyethylene glycol, or fatty acid ester of sorbitan.

In case the formulation of the present invention is the suspension, it may include, as a carrier ingredient, water, a liquid diluent such as ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, or tracant.

In case the formulation of the present invention is a surfactant-containing cleansing agent, it may include, as a carrier ingredient, aliphatic alcohol sulfate, aliphatic alcohol ether sulfate, sulfosuccinic acid monoester, isethionate, imidazolinium derivative, methyl taurate, sarcosinate, fatty acid amide ether sulfate, alkylamidobetaine, aliphatic alcohol, fatty acid glyceride, fatty acid diethanolamide, vegetable oil, linoline derivative, or ethoxylated glycerol fatty acid ester.

In addition to the green tea peptide, the cosmetic composition may further comprise functional additives and ingredients contained in a general cosmetic composition. The functional additives may include ingredients selected from the group consisting of water-soluble vitamins, oil-soluble vitamins, peptides of a high molecular weight, polysaccharides of a high molecular weight, sphingolipids, and seaweed extracts.

In addition to the above functional additives, if necessary, ingredients contained in a general cosmetic composition may be added to the cosmetic composition. Other ingredients that may be added may include oil and fat ingredients, moisturizers, emollients, surfactants, organic and inorganic pigments, organic powders, ultraviolet absorbers, preservatives, disinfectants, antioxidants, plant extracts, pH adjusters, alcohol, pigments, fragrances, blood circulation accelerators, cooling sensitizers, restriction agents, and purified water.

As an example, the present invention can provide the following embodiments.

A first embodiment can provide a peptide containing the amino acid sequence of SEQ ID NO: 1.

A second embodiment can provide the peptide according to the first embodiment, wherein the peptide consists of the amino acid sequence of SEQ ID NO: 1.

A third embodiment can provide a method for improving skin wrinkles, comprising administering an effective amount of a peptide containing the amino acid sequence of SEQ ID NO: 1 to a subject in need thereof.

A fourth embodiment can provide the method according to the third embodiment, wherein the peptide consists of the amino acid sequence of SEQ ID NO: 1.

A fifth embodiment can provide the method according to one or more of the third and fourth embodiments, wherein the peptide suppresses the activity of MMIP-1.

A sixth embodiment can provide the method according to one or more of the third to fifth embodiments, wherein the peptide is formulated in the form of a composition and is contained in an amount of 0.001 to 1% by weight based on the total weight of the composition.

A seventh embodiment can provide the method according to one or more of the third to sixth embodiments, wherein the peptide is formulated in the form of a cosmetic composition or a health food composition.

An eighth embodiment can provide a method for senotherapy, comprising administering an effective amount of a peptide containing the amino acid sequence of SEQ ID NO: 1 to a subject in need thereof.

A ninth embodiment can provide the method according to the eighth embodiment, wherein the peptide consists of the amino acid sequence of SEQ ID NO: 1.

A tenth embodiment can provide the method according to one or more of the eighth and ninth embodiments, wherein the peptide is formulated in the form of a composition and is contained in an amount of 0.001 to 1% by weight based on the total weight of the composition.

An eleventh embodiment can provide the method according to one or more of the eighth to tenth embodiments, wherein the peptide is formulated in the form of a cosmetic composition or a health food composition.

Hereinafter, the contents of the present invention will be described in more detail through Examples and Test Examples. However, these Examples and Test Examples are merely presented to understand the contents of the present invention, and the scope of the present invention is not limited to these Examples and Test Examples. Modifications, substitutions, insertions, etc., which are commonly known in the art, may be performed, and these are also included in the scope of the present invention.

### [Test Example 1] Confirmation of FOXO4 Expression in Skin Senescent Cells

In order to confirm whether FOXO4 expression is increased in skin senescent cells, the presence or absence of the FOXO4 expression in normal skin fibroblasts and aged skin fibroblasts was identified through immunofluorescence (IF) stain and polymerase chain reaction (PCR) experiments. The aged skin fibroblasts were prepared with a method of inducing early senescence (DI model) by treating Doxorubicin (Dox) and Insulin-Like Growth Factor-1 (IGF-1) on the normal skin fibroblasts and a method of inducing senescence through continuous subculture of the skin fibroblasts (RS model).

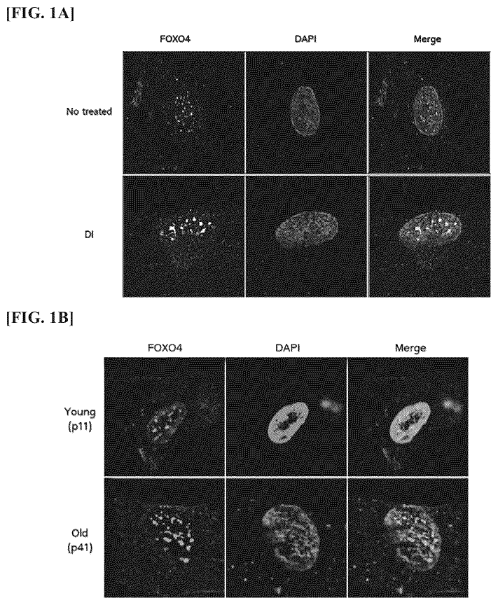

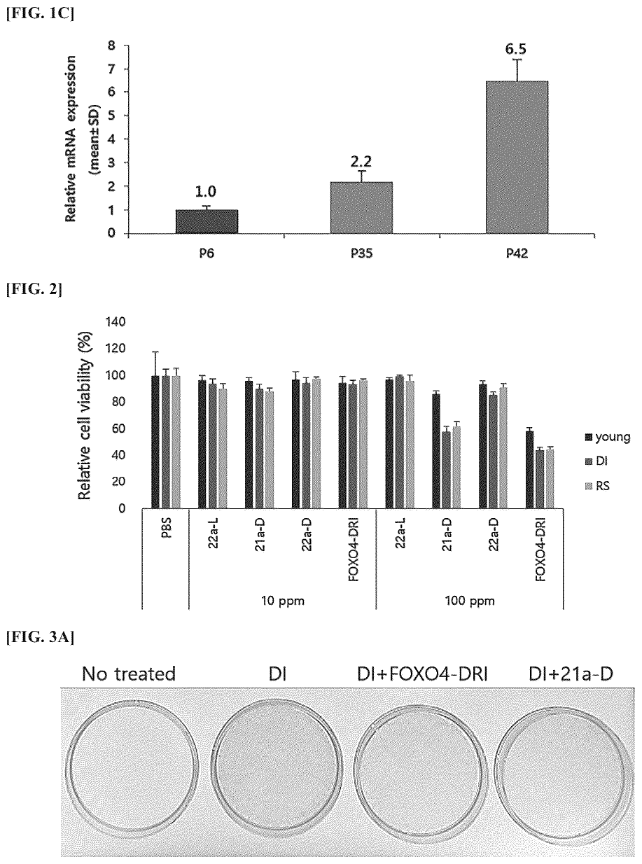

Specifically, in the case of the DI model, the skin fibroblasts (Normal human dermal fibroblasts, Lonza, Switzerland) were cultured in DMEM Medium (Lonza, Switzerland) containing 10% FBS, and then were inoculated into a chamber slide (Thermo Fisher Scientific, USA) at 1×10³ cells per well and cultured in a CO₂ incubator of 5% concentration at 37° C. for 24 hours. The cultured wells were treated with each of Doxorubicin-hydrochloride (Sigma) and IGF-1 (Sigma) in a concentration of 100 ng/ml for 4 days to induce early senescence of the skin fibroblasts. As a control group, the normal skin fibroblasts that were not treated with Dox or IGF-1 were prepared. Thereafter, for the immunofluorescence stain, the cell culture solution was washed with PBS three times and fixed in 4% paraformaldehyde for 30 minutes. Then, the skin fibroblast sample was washed with PBS containing 0.2% Triton X-100 three times, added to PBS containing 3% BSA, and left for 1 hour. Next, the sample was incubated in 1% BSA containing rabbit polyclonal anti-FOXO4 antibody (SAB4501887, Sigma) (1:500 dilution) at 4° C. overnight to allow the antibody to bind to an antigen contained in the skin fibroblasts. Thereafter, the Sample was washed with the PBS three times, incubated with Alexa Fluor®488-conjugated goat polyclonal secondary antibody (A32731, ThermoFisher Scientific) (1:1000 dilution) against rabbit IgG-H&L at a room temperature for 1 hour, and then washed with the PBS three times. Next, a drop of the mounting solution containing DAPI was placed on a microscope slide glass and fixed by raising the coverslip itself. The fixed sample was observed by a confocal microscope (LSM 980, Zeiss, Germany). The results were shown in FIG. 1A.

In the case of the RS model, the aged skin fibroblasts were prepared by continuously culturing skin fibroblasts (Normal human dermal fibroblasts, Lonza, Switzerland) in DMEM Medium (Lonza, Switzerland) containing 10% FBS over 30 to 40 times. For the immunofluorescence staining test, each of the aged skin fibroblasts (p41) and the normal skin fibroblasts was inoculated into a chamber slide (Thermo Fisher Scientific, USA) at 1×10³ cells per well, and cultured in a CO₂ incubator of 5% concentration at 37° C. for 48 hours. Thereafter, the cell culture solution was washed with PBS three times and fixed in 4% paraformaldehyde for 30 minutes. Next, the skin fibroblast sample was washed with PBS containing 0.2% Triton X-100 three times, added to PBS containing 3% BSA, and left for 1 hour. Then, the sample was incubated in 1% BSA containing a rabbit polyclonal anti-FOXO4 antibody (SAB4501887, Sigma) (1:500 dilution) at 4° C. overnight to allow the antibody to bind to an antigen contained in the skin fibroblasts. Thereafter, the sample was washed with the PBS three times, incubated with Alexa Fluor®488-conjugated goat polyclonal secondary antibody (A32731, ThermoFisher Scientific) (1:1000 dilution) against rabbit IgG-H&L at a room temperature for 1 hour, and then was washed with the PBS three times. Next, a drop of the mounting solution containing DAPI was placed on a microscope slide glass, and fixed by raising the coverslip itself. The fixed sample was observed by a confocal microscope (LSM 980, Zeiss, Germany). The results were shown in FIG. 1B.

Further, a change in the expression level of FOXO4 depending on the number of subcultures of the skin fibroblasts was identified through a PCR test. The skin fibroblasts subcultured 6, 35, and 42 times, respectively, were inoculated into a 6-well plate at 1×10⁶ cells per well, and cultured in a 5% CO₂ incubator at 37° C. for 48 hours. Thereafter, the cell culture solution was washed with the PBS three times, and total RNA was isolated using a trizol solution (TRIzol™ Reagent, Invitrogen) extraction method. 1 g of the isolated RNA was synthesized into cDNA using reverse transcriptase (RevertAid Firist strand cDNA synthesis kit, Thermofisher), and then diluted to a final concentration of 200 μl. In order to perform PCR, a mixing kit was prepared by mixing 1 μl of FOXO4 primer (Hs00172973_m1, ThermoFisher Scientific), 10 μl of Taqman universal master mix II with UNG (Applied Biosystems), and 5 μl of distilled water to allow the mixture to be diluted to a final volume of 16 μl. 16 μl of the mixing kit and 4 μl of cDNA were mixed to perform qPCR under the following reaction conditions. The reaction conditions were 95° C., initialization reaction for 10 minutes, denaturation at 95° C. for 15 seconds, annealing at 60° C. for 60 seconds, and extension reaction at 50° C. for 20 seconds, which were set to be performed in a PCR device (7500 Fast Real-time PCR system, Applied Biosystems) with repetition of 40 times. An expression level of the target gene was made through correction for a GAPDH gene, and the results were shown in FIG. 1C.

As shown in FIGS. 1A, 1B, and 1C, the expression of FOXO4 could be confirmed to increase in the aged skin fibroblast model (DI (Doxorubicin/IGF-1) treatment model and RS (replicative senescence) model). In addition, it could be confirmed from the results of FIG. 1C that in the case of the RS model, the expression of FOXO4 significantly increased at passages 35 to 42.

### [Test Example 2] Confirmation of Selective Apoptosis Efficacy on Skin Senescent Cells

As in Test Example 1, after the skin fibroblasts (DI model) that induced senescence by treating with each of Doxorubicin and IGF-1 in a concentration of 100 ng/ml for 4 days or the fibroblasts (RS model) aged by culturing the skin fibroblasts up to passage 40 were treated with a peptide (21a-D) consisting of the amino acid sequence of AYSQNGWANRRRRGGRRRRRR (SEQ ID NO: 1), a peptide (22a-L) consisting of the amino acid sequence of QRKKRRQRRRPRRNAWGNQSYA (SEQ ID NO: 2), a peptide (22a-D) consisting of the amino acid sequence of AYSQNGWANRRPRRRQRRKKRG (SEQ ID NO: 3), and a peptide (FOXO4-DRI) consisting of the amino acid sequence of LTLRKEPASEIAQSILEAYSQNGWANRRSGGKRPPPRRRQRRKKRG (SEQ ID NO: 4) in each concentration of 100 μg/ml for 3 days, a survival rate of the aged cells was measured. The survival rate of aged cells was measured using a CCK-8 quantitative kit (cell counting kit-8, Dojindo) according to the proposed protocol. Specifically, after treating the CCK-8 with a DMEM medium diluted to 1/100, at the point of time 1 hour has lapsed, an absorbance was measured at 450 nm using spectrostar nano (BMG labtech). The results were shown in FIG. 2.

As shown in FIG. 2, it could be confirmed that the selective apoptosis of only skin senescent cells was induced when treated with the peptide 21a-D of the amino acid sequence of SEQ ID NO: 1 according to an embodiment of the present invention. When treated with the peptides 22a-L and 22a-D of the amino acid sequences of SEQ ID NOS: 2 and 3, the apoptosis effect of senescent cells was minimal. In the case of the peptide FOXO4-DRI of the amino acid sequence of SEQ ID NO: 4, it was confirmed that the apoptosis effect had on the senescent cells as well as even on the normal cells, which means that the selective apoptosis effect on the senescent cells was confirmed to be weak. It could be confirmed from the above that the peptide having the amino acid sequence of SEQ ID NO: 1 according to an embodiment of the present invention exhibits the selective apoptosis effect on the skin senescent cells.

### [Test Example 3] Visual Observation of Selective Apoptosis Efficacy on Skin Senescent Cells

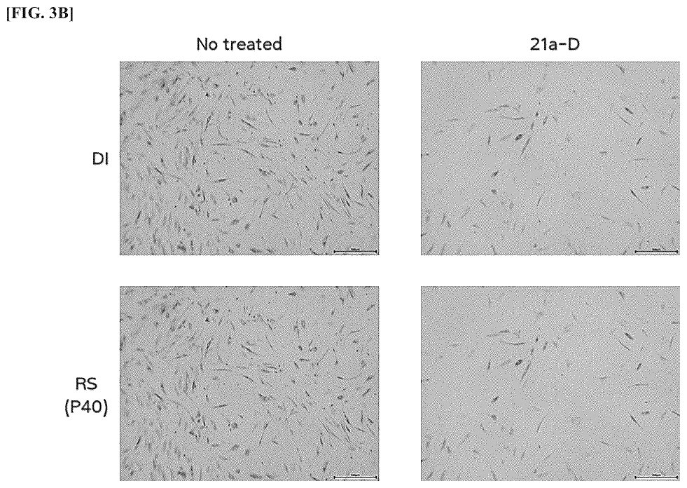

In the same manner as that of Test Example 2, after the DI model and the RS model were treated with a peptide (21a-D) consisting of the amino acid sequence of AYSQNGWANRRRRGGRRRRRR (SEQ ID NO: 1) and a peptide (FOXO4-DRI) consisting of the amino acid sequence of LTLRKEPASEIAQSILEAYSQNGWANRRSGGKRPPPRRRQRRKKRG (SEQ ID NO: 4), SA-3-gal (senescent associated-β-galactosidase) stain and microscopic observation were performed to visually confirm an apoptosis efficacy of the skin senescent cells. The SA-3-Gal stain was performed according to the protocol using the Senescence Cell Histochemical Staining Kit (CS0030, Sigma, USA), and after 24 hours of the staining, it was confirmed whether or not the cells were stained through an optical microscope. The results were shown in FIGS. 3A and 3B.

As shown in FIGS. 3A and 3B, it could be confirmed that the number of senescent cells was significantly reduced when treated with a peptide having the amino acid sequence of SEQ ID NO: 1 according to an embodiment of the present invention.

### [Test Example 4] Observation of Cytoplasmic Movement of p53

If FOXO4 (Forkhead box protein 4) receives an external stimulus such as an ultraviolet ray, it binds to p53 at a DNA damage site to cause cell senescence through the expression of p21, a cellular senescence regulator. If FOXO4-p53 binding is inhibited, p53 moves to mitochondria and induces a release of cytochrome c to cause apoptosis of the senescent cells.

It was confirmed through the immunofluorescence (IF) stain experiment whether or not this phenomenon was observed when the skin senescent cells were treated with the peptide having the amino acid sequence of SEQ ID NO: 1 according to an embodiment of the present invention. The aged skin fibroblasts treated with DI (Doxorubicin/IGF-1) were treated with the peptide 21a-D in the same manner as in Test Example 2, and then the expression and cytoplasmic movement of FOXO4 and p53 were observed.

Specifically, the skin fibroblasts were inoculated into a chamber slide (Thermo Fisher Scientific, USA) at 1×10³ cells per well and cultured in a 5% CO₂ incubator at 37° C. for 24 hours. The cultured wells were treated with each of Dox and IGF-1 in a concentration of 100 ng/ml for 4 days to induce early senescence of the skin fibroblasts, and then treated with the peptide 21a-D in a concentration of 100 ug/ml for 3 days followed by being further cultured. For immunofluorescence stain, the cell culture solution was washed with PBS three times and fixed by treating it with ice-cold methanol at −20° C. for 10 minutes. Next, it was added into PBS containing 3% BSA and left for 1 hour. Next, the sample was incubated in in 1% BSA containing a rabbit polyclonal anti-FOXO4 antibody (SAB4501887, Sigma, USA) (1:500 dilution) and a mouse anti-p53 antibody (#9286, Cell signaling technology, USA) (1:400 dilution), respectively, at 4° C. overnight to allow the antibody to bind to an antigen contained in the skin fibroblasts. Thereafter, the sample was washed with the PBS three times, and reacted with Alexa Fluor®488-conjugated goat polyclonal secondary antibody (A32731, ThermoFisher, USA) (1:1000 dilution) against rabbit IgG-H&L and Alexa Fluor®488-conjugated goat polyclonal antibody (A11001, ThermoFisher, USA) (1:1000 dilution) against mouse IgG-H&L at a room temperature for 1 hour, and then washed with the PBS three times. Next, a drop of the mounting solution containing DAPI was placed on a microscope slide glass, and fixed by raising the coverslip itself. The fixed sample was observed by a confocal microscope (LSM 980, Zeiss, Germany). The results were shown in FIG. 4.

As shown in FIG. 4, it could be confirmed that the expression of FOXO4 and p53 increased in the nucleus of the skin senescent cells through DI treatment, and that p53 moved to a cytoplasm in the nucleus by treatment with the peptide 21a-D to suppress binding to FOXO4. It was confirmed from the above that the treatment with the peptide having the amino acid sequence of SEQ ID NO: 1 according to an embodiment of the present invention suppressed the binding of p53 and FOXO4 in the nucleus, thereby inducing apoptosis of the skin senescent cells.

### [Test Example 5] Confirmation of MMP-1 Suppressive Activity

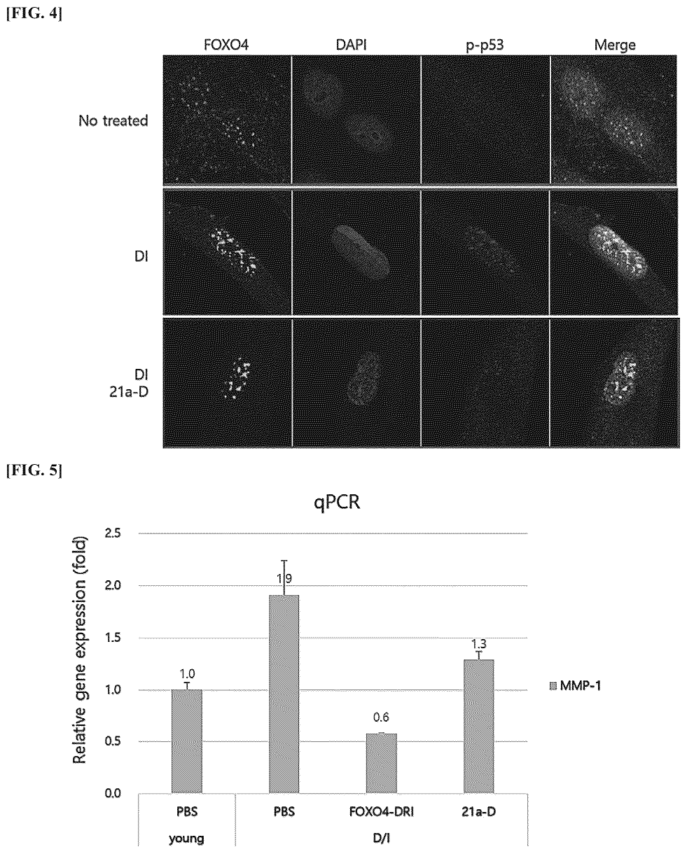

After treating the DI model with the peptide (21a-D) consisting of the amino acid sequence of AYSQNGWANRRRRGGRRRRRR (SEQ ID NO: 1) and the peptide (FOXO4-DRI) consisting of the amino acid sequence of LTLRKEPASEIAQSILEAYSQNGWANRRSGGKRPPPRRRQRRKKRG (SEQ ID NO: 4) in the same manner as in Test Example 2 above, an ability to suppress the expression of MMP-1 in the senescent cells was identified. After recovering the cultured cells, total RNA was isolated using a TRIzol™ solution (TRIzol™ Reagent, Invitrogen) extraction method. 1 μg of the isolated RNA was synthesized into cDNA using reverse transcriptase (RevertAid Firist strand cDNA synthesis kit, Thermofisher), and then diluted to a final concentration of 200 μl. In order to perform PCR, a mixing kit was prepared by mixing 1 of MMP-1 primer (Hs00899658_m1, ThermoFisher Scientific), 10 μl of Taqman universal master mix II with UNG (Applied Biosystems), and 5 μl of distilled water to allow the mixture to be diluted to 16 μl. 16 μl of the mixing kit and 4 μl of cDNA were mixed to perform qPCR under the following reaction conditions. The reaction conditions were 95° C., initialization reaction for 10 minutes, denaturation at 95° C. for 15 seconds, annealing at 60° C. for 60 seconds, and extension reaction at 50° C. for 20 seconds, which were set to be performed in a PCR device (7500 Fast Real-time PCR system, Applied Biosystems) with repetition of 40 times. An expression level of the target gene was made through correction for a GAPDH gene, and the results were shown in FIG. 5.

As shown in FIG. 5, it could be confirmed that the expression of MMP-1 increased in the skin senescent cells through DI treatment, and that the expression of MMP-1 was suppressed upon treatment of the peptide (21a-D) having the amino acid sequence of SEQ ID NO: 1 according to an embodiment of the present invention.

What is claimed is:

1. A peptide containing the amino acid sequence of SEQ ID NO: 1.

2. The peptide according to claim 1,
wherein the peptide consists of the amino acid sequence of SEQ ID NO: 1.

3. A method for improving skin wrinkles, comprising administering an effective amount of a peptide containing the amino acid sequence of SEQ ID NO: 1 to a subject in need thereof.

4. The method according to claim 3,
wherein the peptide consists of the amino acid sequence of SEQ ID NO: 1.

5. The method according to claim 3,
wherein the peptide suppresses the activity of MMP-1.

6. The method according to claim 3,
wherein the peptide is formulated in the form of a composition and is contained in an amount of 0.001 to 1% by weight based on the total weight of the composition.

7. The method according to claim 3,
wherein the peptide is formulated in the form of a cosmetic composition or a health food composition.

8. A method for senotherapy, comprising administering an effective amount of a peptide containing the amino acid sequence of SEQ ID NO: 1 to a subject in need thereof.

9. The method according to claim 8,
wherein the peptide consists of the amino acid sequence of SEQ ID NO: 1.

10. The method according to claim 8,
wherein the peptide is formulated in the form of a composition and is contained in an amount of 0.001 to 1% by weight based on the total weight of the composition.

11. The method according to claim 8,
wherein the peptide is formulated in the form of a cosmetic composition or a health food composition.


SEQUENCE LISTING (4 sequences)

SEQ ID NO: 1 - protein, 21 aa, unidentified
AYSQNGWANRRRRGGRRRRRR

SEQ ID NO: 2 - protein, 22 aa, unidentified
QRKKRRQRRRPRRNAWGNQSYA

SEQ ID NO: 3 - protein, 22 aa, unidentified
AYSQNGWANRRPRRRQRRKKRG

SEQ ID NO: 4 - protein, 46 aa, unidentified
LTLRKEPASEIAQSILEAYSQNGWANRRSGGKRPPPRRRQRRKKRG